(19) United States
(12) Patent Application Publication
(10) Pub. No.: US 2026/0331266 A1
(43) Pub. Date: Oct. 22, 2026

(54) AI-ASSISTED HOSPITAL ASSET TRACKING

(71) Applicant: Peritas AI Inc., Fort Lauderdale, FL (US)

(72) Inventors: Martin Roche, Fort Lauderdale, FL (US); Joseph DeCerce, Fort Lauderdale, FL (US)

(21) Appl. No.: 19/566,707

(22) Filed: Mar. 13, 2026

Related U.S. Application Data
(63) Continuation-in-part of application No. 19/431,214, filed on Dec. 23, 2025.
(60) Provisional application No. 63/782,696, filed on Apr. 3, 2025.

(51) Int. Cl. G16H 40/20

(52) U.S. Cl. CPC G16H 40/20

(57) ABSTRACT

A database system contains data collected from images and operations in a hospital. A machine learning (ML) model is iteratively trained on data in the database system to control one or more robots to convey plural assets in the hospital to operating rooms for use in an operation.

### FIELD

The present application relates generally to AI-assisted hospital asset tracking.

### BACKGROUND

Workflows prior to, during, and after surgeries such as knee replacement surgeries can be complex.

### SUMMARY

As understood herein, prior to surgery, for example, the necessary surgical instruments or tools required by the attending surgeon for a particular procedure must be sterilized, assembled into a tray, the tray sterilized, and then transported to the operating room (OR) in time for the scheduled surgery. This requires significant coordination with OR cleaning and the provision of additional hospital assets such as patient implants including prosthetics, patient gurneys, etc. During surgery, complex operations dynamically require the adherence to procedures and flexibility to respond to unforeseen events, such as the dropping of an instrument which then must be replaced by a sterile substitute. Modifications to the workflow itself might be required. Post-surgery, assets must be returned to the appropriate places in the hospital and tools re-sterilized for subsequent use. Present principles understand that artificial intelligence (AI) technologies may be highly advantageous for coordinating this process end-to-end, learning with each procedure how to improve overall patient service.

Accordingly, in one aspect an apparatus includes a database system containing data collected from images and operations in at least one hospital. The apparatus also includes at least one computer storage storing at least one machine learning (ML) model executable by at least one processor assembly and iteratively trained on data in the database system to control one or more robots to convey plural assets in the hospital to at least one operating room for use in an operation.

In some embodiments the asset includes a tray supporting surgical tools, or an implant such as a prosthetic, or a patient gurney, or other asset.

In example implementations the ML model is configured to control the robot based at least in part on an identity of a surgeon associated with the operation. Additionally the ML model can be configured to control the robot based at least in part on a type of surgery the operation is. Yet again, the ML model ma be configured to control the robot based at least in part on a time and date of the operation.

The apparatus may further include plural tracking readers mounted in the hospital to detect presence of assets and transmit indication thereof to the database system. The tracking readers can detect respective wireless tracking tags associated with respective assets. If desired, the tracking readers can include Bluetooth readers.

In another aspect, a method includes autonomously navigating at least one robot through perioperative areas in a hospital, and using at least one machine learning (ML) model, analyzing historical asset usage data and operations to predict upcoming asset demand. The asset also includes identifying and tracking assets in the hospital using an asset identification technology to obtain location data for each asset identified by the ML model for upcoming asset demand. The method may include retrieving the assets according to ML model output and conveying the assets to an operating room using the robot, and transmitting asset usage events in the operating room to a database system.

In another aspect, an apparatus includes a robot and at least one processor assembly configured to execute a machine learning (ML) model to control the robot to move through a hospital to an operating room in which an operation is to be conducted. A database system stores information regarding assets in the hospital. The processor assembly is configured to communicate with the database system to control the robot to move to one or more locations at which respective assets required for the operation are located according to the database system and to engage one or more of the assets for conveyance thereof. A sensing system detects presence of the assets as the robot moves them through the hospital to the operating room.

The details of the present application, both as to its structure and operation, can be best understood in reference to the accompanying drawings, in which like reference numerals refer to like parts, and in which:

### BRIEF DESCRIPTION OF THE DRAWINGS

FIG. 1 is a block diagram of an example system in accordance with present principles;

FIG. 2 illustrates example overall logic in example flow chart format;

FIG. 3 illustrates an example data structure consistent with present principles;

FIG. 4 illustrates a tool tray on an assembly table;

FIG. 5 illustrates the tool tray of FIG. 4 being filled with surgical tools by a robot;

FIG. 6 illustrates example logic in example flow chart format for training an ML model to control the robot of FIG. 5;

FIG. 7 illustrates a block diagram of the example proximity tag of the tray of FIG. 4;

FIG. 8 illustrates example logic in example flow chart format for configuring a tray with tools;

FIG. 9 illustrates example logic in example flow chart format for tracking surgical tool trays and the carts on which they are transported;

FIG. 10 illustrates a cart with plural tool trays along with tracking instrumentation;

FIG. 11 illustrates an ML model receiving input to output case configuration;

FIG. 12 illustrates a UI for tracking inventory status for an operation;

FIG. 13 illustrates example logic in example flow chart format for tracking assets as they move within a hospital;

FIG. 14 illustrates example logic in example flow chart format for training the ML model of FIG. 11;

FIG. 15 schematically illustrates a robot and related operating room (OR) personnel and equipment;

FIG. 16 schematically illustrates an OR;

FIG. 17 illustrates example overall logic in example flow chart format for data gathering related to a surgical operation in the OR;

FIG. 18 is a block diagram of components of the robot shown in FIGS. 15 and 16;

FIG. 19 illustrates a surgeon and robot performing surgery;

FIG. 20 illustrates processing components for controlling the robot in the OR;

FIG. 21 illustrates example logic in example flow chart format for generating and maintaining the 3D map in FIG. 20;

FIG. 22 illustrates example logic in example flow chart format for machine vision-to-control of the robot; and

FIG. 23 illustrates, in example flow chart format, details of logic executed by the predictive workflow model from FIGS. 18 and 20.

### DETAILED DESCRIPTION

This disclosure relates generally to computer ecosystems including aspects of artificial intelligence (AI) assistance for hospital workflows.

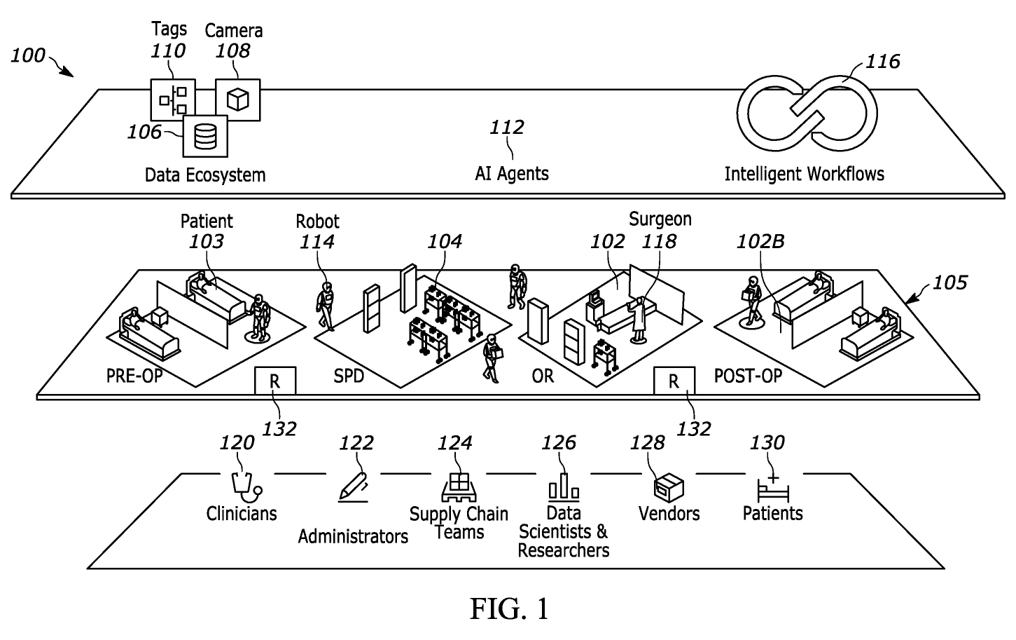

FIG. 1 illustrates a hospital 100 which includes one or more operating rooms (OR) 102 and at least one sterile processing department (SPD) 104. As illustrated in FIG. 1, pre-operation procedures may include preparation of a patient 103 in a room and may further include including sterilizing the OR and transporting hospital assets to the OR, including trays of sterile surgical instruments (also referred to herein as “tools”) from the SPD 104 to the OR 102. A robot may hand a tool from the tray to a human or robotic surgeon. An operation is then performed in the OR 102 on the patient, after which, as indicated at 102B, post-operation transport and care of the patient 103 to recovery 105 may be performed along with cleanup of the OR and transport of assets back to the appropriate location (such as used tools to the SPD 104, etc.)

In accordance with present principles, to aid in the end-to-end process shown in FIG. 1, a database system 106 stores and correlates data from various sources, including cameras 108 and tracking tags 110, described more fully below along with other sensors including haptic feedback sensors. One or more AI agents 112, also referred to herein as machine learning (ML) models, access the database system 106 and outputs of the cameras 108 and tags 110 to control one or more mobile robots 114, which may be humanoid robots with appropriate end effectors for completing specific manipulative tasks, according to training of the ML models 112 to implement workflows 116 related to pre-op, surgical, and post-op tasks. The workflows 116 may be computer-stored in the database system 106 and may evolve over time as one or more ML models iteratively or recursively learn improvements in the workflows.

In this way, AI-controlled robots 114 can assist in surgeries that may involve one or more humans 118. For example, the robots 114 may assist clinicians 120, administrators 122, supply chain personnel 124, scientists and researchers 126, vendors 128, and patients 130.

One or more of the cameras 108 may be located in the SPD 104 for imaging plural sterilized surgical instruments output by a sterilization assembly such as an autoclave in the SPD. One or more cameras 108 also may be located in the OR 102 to image the operation, including human and robotic activity and medical machines that display data, which may then be classified by, e.g., a machine learning classifier. Tags 110, which may be near field wireless proximity tags as described elsewhere// herein, may be attached to hospital assets including tool trays, transport carts, gurneys, prosthetics, etc. and may be tracked by fixed readers 132 to track assets and upload time-stamped location data of the assets to the DB system 106. The tags thus can be read to provide unique identifiers of the assets to which they are attached, and tray tags may be correlated with the tools loaded onto the respective trays and confirmed using machine vision. Note that the robot 114 also may include its own tag reader so that as it moves a tray it can read the tray ID and send the tray ID along with location to the database. In this way the robot 114 may function as a mobile reader that can also communicate with other proximity tags and readers and with the database 106. Because it may include its own proximity reader, the robot 114 may, in response to a command to retrieve a particular instrument, access the database to determine the approximate location of the instrument (usually in a tray on a cart) and navigate to the appropriate cart to retrieve the appropriate tray using its proximity reader to read the tag associated with the correct tray.

Further details of AI assisting in loading trays, tracking assets, and assisting in the theater of the OR are set forth further below.

In an initial example, details of which are discussed further below, the robot 114 in the SPD 104, which may be a humanoid robot, can identify and place surgical instruments in tray in accordance with IFU, particular intended surgery, and surgeon preferences downloaded by the robot from the data center. The robot can then confirm, using, e.g., machine vision, that the tray is correctly loaded with appropriate instruments. The tray is then sterilized and placed by the robot on racks of carts with readers that can read the tray tags and upload proximity signals to the database to upload to the database continuous indication of which carts contain which trays (and hence which tools) and where the carts (and trays) are currently located.

The cart alluded to above may be autonomous on its own or pushed by a robot. As the cart navigates through the hospital using, e.g., cameras, proximity sensors, and an electronic map, it passes various readers mounted on strategic locations in halls and doorways which read the tag of the cart to track in real time the location of the cart and its contents. The real time location of each asset in the hospital is logged into the database and can be accessed by hospital personnel and other robots.

In this way, an OR team is kept aware of location and timing for bringing the cart. As a result, during a procedure in the OR 102, should the surgeon modify the surgery and require a different instrument or implant, a robot can scan the OR using a camera on the robot and using machine vision recognize that the required tool is missing. The robot may communicate via the hospital network with another robot to locate and bring the required instrument tray to the OR. Alternatively, the robot in the OR may depart the OR and retrieve the required instrument itself using instrument location information in the database 106.

Any one or more of the robots herein may not only complete tasks responsive to orders from OR personnel or downloaded from the network but may also analyze and store data, communicating through the hospital network with other robots and humans on the surgical team and transmitting data to the database 106. This enables the robot to be a communicator, task enabler, and mobile control center.

In light of this and further disclosure below, the instant application discloses a living, real-time dynamic system that is proactive and efficiently reactive as conditions change. As recognized herein, surgery is a very dynamic process that, without present principles, reacts in a step-wise fashion because no parallel systems are built, since doing so would require real time data and an actionable workforce.

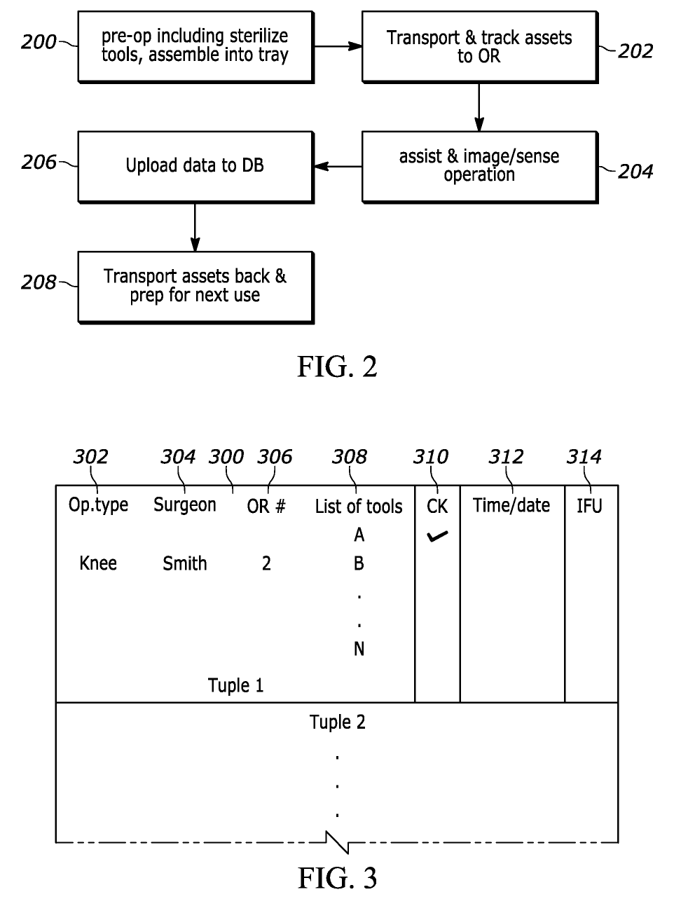

FIG. 2 illustrates the process above. State 200 indicates the pre-op processes are undertaken including sterilizing required tools and assembling them into trays in the SPD. State 202 indicates that the trays, which may be loaded onto carts, are transported and tracked to the OR. Other assets including prostheses and patient gurneys may be transported under robotic control to the OR.

State 204 indicates that during the operation the AI agents may control one or more robots to assist, such as by handing surgeons required tools from the tray or indicating that a tool may have been rendered unsterilized because, e.g., it has been dropped. A robot may retrieve as substitute tool for a dropped tool. The procedure may be imaged by one or more of the cameras 108 and haptic sensors, e.g., on the robot may be uploaded with the images and correlated to the images to the DB system 106 at state 206. Among other things a 3D spatial map of the operating room may be generated based on imaging people and objects during the procedure.

Post-op, state 208 indicates that the assets are returned to their appropriate locations in the hospital and prepared for their next use.

FIG. 3 illustrates a portion of a data structure that may be contained in the database system 106. An example database system 106 may be implemented by a relational database system that includes plural tuples 300. In the non-limiting example shown each tuple 300 may relate an operation type 302 to a particular surgeon 304 and a particular OR 306. The DB 106 may also include in a tuple a list of surgical tools 308 preferred by the surgeon for the particular operation. The DB 106 may include an indication 310 indicating whether sterilization of the tools has been satisfactorily checked, times and dates 312 of one or more upcoming operations, and instructions for use (IFU) 314 related to the tools 308, which can include arrangements of the tools in a tray, sterilization requirements, etc. Additional data discussed herein may be contained in the database system. Database systems other than relational databases may be used.

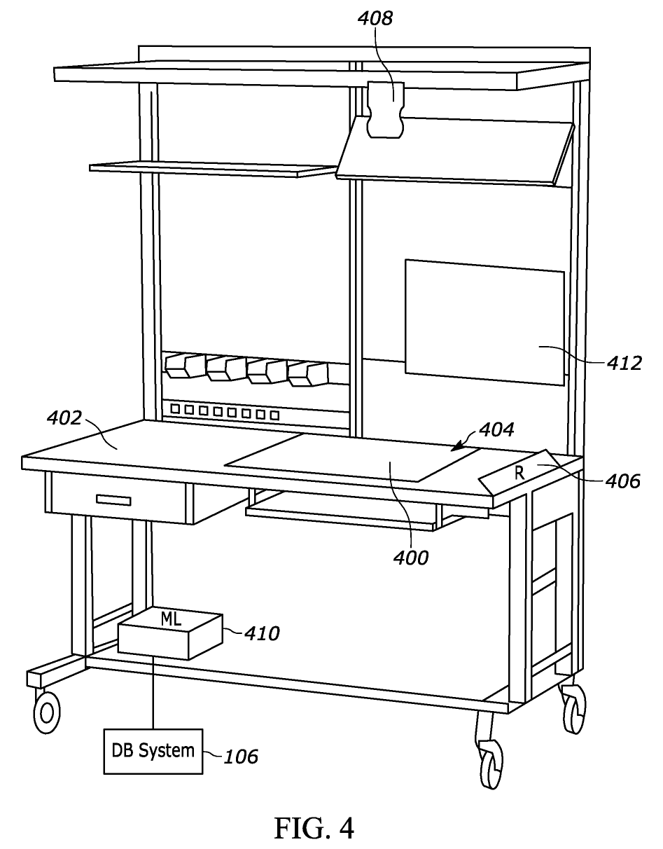

FIG. 4 illustrates aspects of state 200 in FIG. 2. An instrument tray 400 may be placed by a human or a robot on an assembly bench 402. A tag 404 such as a near field proximity tag may be engaged with the tray 400. Tags may be referred to herein as “trackers”. The tag 404 may be, e.g., a magnetic NFC device such as a Rubee^{TM} tag or a radiofrequency (RF) NFC device such as a Bluetooth^{TM}-6 or other appropriate device, and may be configured to withstand autoclaving or other sterilization process. A Rubee tag may conform to IEEE 1902.1. 4G GNSS also may be used.

A reader or receiver 406, which may be considered to be a “router” that communicates with a network including the DB system 106, may be mounted on or near the assembly bench 402 to detect the presence of the tag 404. The tag 404 indicates a unique identifier of the tray 400. The reader 406 may be in wired or wireless communication with the database system 106 to communicate the reader location and the tray identifier to the DB system 106 such that the DB system 106 knows the current location of the tray 400.

One or more cameras may be positioned to image tools on the tray 400. The images from the camera 408 may be provided to one or more machine learning (ML) models 410 which may be, e.g., implemented by a processor assembly associated with the assembly bench 402, or by a robot who may be placing tools on the tray 400 in accordance with instructions for use (IFU) described herein, or by a processor assembly associated with the DB system 106 or hospital network or server, etc. The ML model 410, which may be, e.g., a linear classifier or a convolutional neural network (CNN), classifies the tools based on the images and sends the classifications to the DB system 106, which associates the now-classifier tools with the identified tray 400. One or more displays 412 may be associated with the bench 402 for presenting IFU to a human assembler. Once the tools are arranged on the tray including a pattern of arrangement defined by the IFU, a final image may be taken and classified to determine conformity of the tool types and arrangement with the IFU. If IFU conformity is not indicated an error message may be presented on the display 412

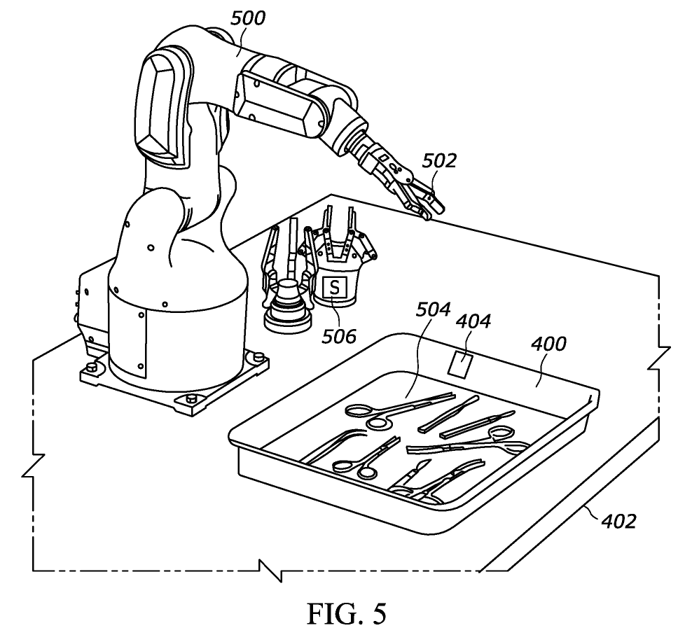

FIG. 5 illustrates that a robot 500 with one or more removable end effectors 502 may be used to arrange surgical tools 504 on the tray 400. The end effectors 50 may be tailored to handling specific tools 504 and may include respective force sensors 506 to provide haptic feedback signals that may be sent by the robot 500 to the DB system 106 for integration with other indicia related to the tools 504.

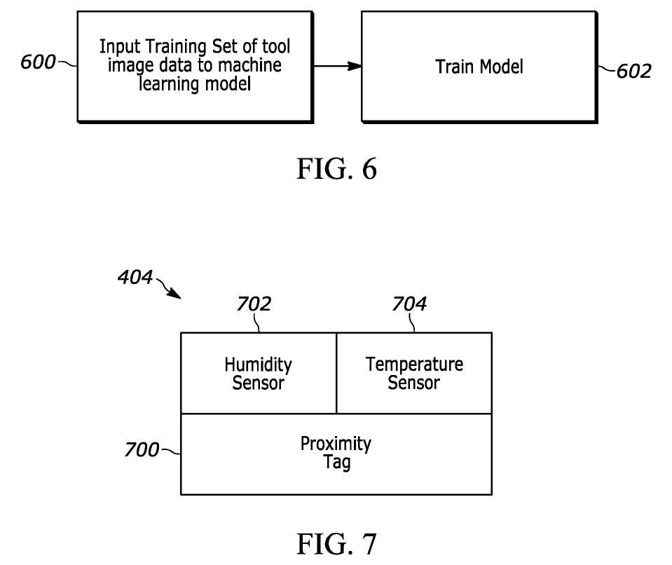

FIG. 6 illustrates example training logic for the ML model 410 shown in FIG. 4. At state 600 an input set of data representing images of various tools is input to the model along with ground truth indication of the tool types. Data may represent the size, shape, and distinguishing characteristics of the tools. Data representing plural images of each tool may be input, with the images being taken from respective different perspectives. At state 602 the model is trained on the images to be able to classify subsequent image data.

FIG. 7 illustrates that the tag 404 may include a proximity tag 700 proper (i.e., the element that indicates to a nearby reader the ID of the tray) as well as a humidity sensor 702 and a temperature sensor 704.

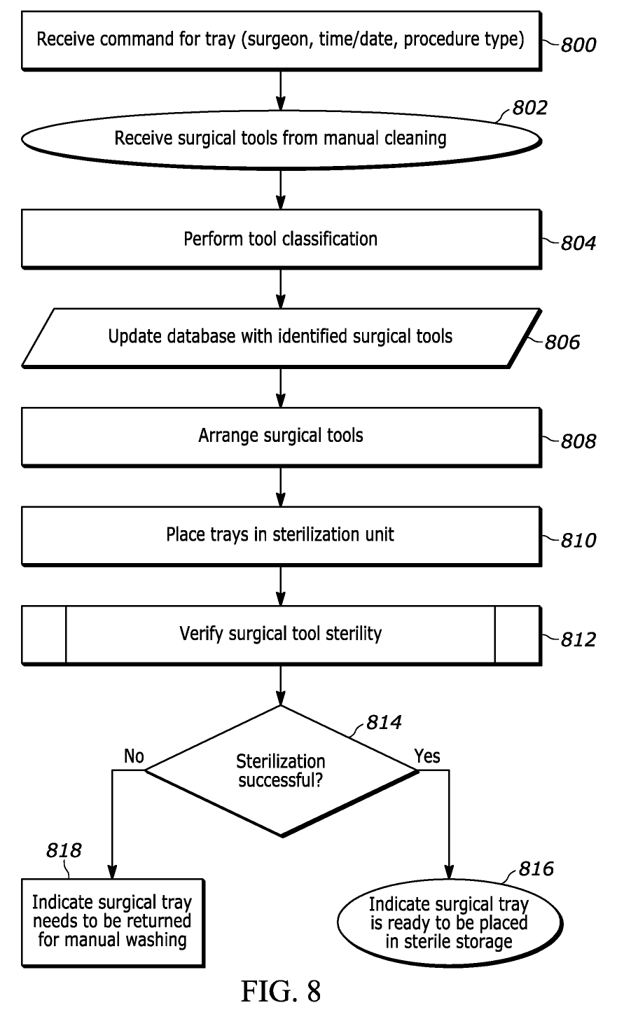

Now refer to FIG. 8. Commencing at state 800, a command is received by a human or robotic assembler to arrange surgical tools on a tray. The command may be presented on the display 412 in FIG. 4 or it may be transmitted to an assembly robot over a computer network. The command may be based, e.g., on a type of operation to be performed, the surgeon/surgeon preferences for tools for that operation, and IFU.

Moving to state 802, a human or robotic assembler receives sterilized tools from sterilization. Proceeding to state 804, the tools are classified using the ML model 410 in FIG. 4 based on images of the tools from the camera 408. If desired, at state 806 the DB system may be updated with the classifications of the tools and the facts that they are located in the SPD and have been sterilized.

Moving to state 808 the assembler arranges tools on the tray 400 in accordance with the IFU provided to the assembler electronically or visually. Once the tools have been correctly selected and assembled on the tray, state 810 indicates that the tray is transported by a robot or human to a sterilizing machine such as an autoclave in which the tray is “blue-wrapped” to contain the tools on the tray.

State 812 indicate that proper sterility is verified. In one example this may be done using signals from the temperature sensor 704 of the tag in FIG. 7. For example, a maximum temperature of at least a threshold temperature may be required to verify correct sterility.

State 814 indicates that a determination is made as to correct sterility, and if correct sterility is verified the process moves to state 816 to so indicate to the DB system and to move the tray to sterile storage. Sterilization dates and methods may accompany the report to the DB system. On the other hand, if sterility is not verified the process moves from state 814 to state 818 to so indicate and to re-sterilize appropriate components.

As may now be appreciated, the robot 114 in the SPD 104 (robot 500 in FIG. 5) can identify and place surgical instruments in tray in accordance with IFU, particular intended surgery, and surgeon preferences downloaded by the robot from the data center. The robot can then confirm, using, e.g., machine vision, that the tray is correctly loaded with appropriate instruments. The tray is then sterilized and placed by the robot on racks of carts with readers that can read the tray tags and upload proximity signals to the database to upload to the database continuous indication of which carts contain which trays (and hence which tools) and where the carts (and trays) are currently located.

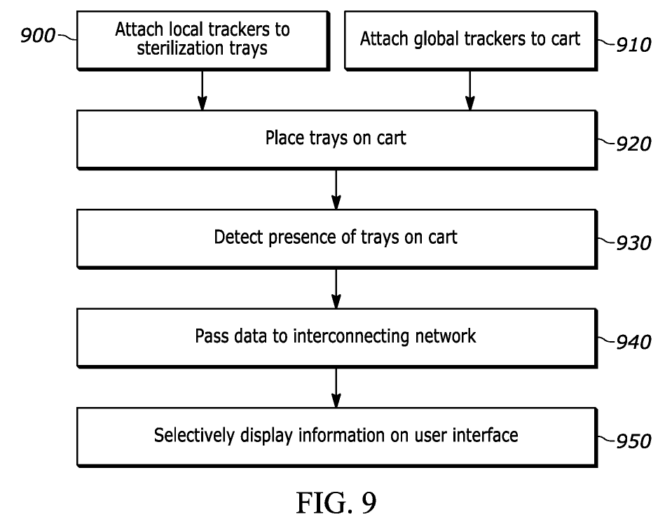

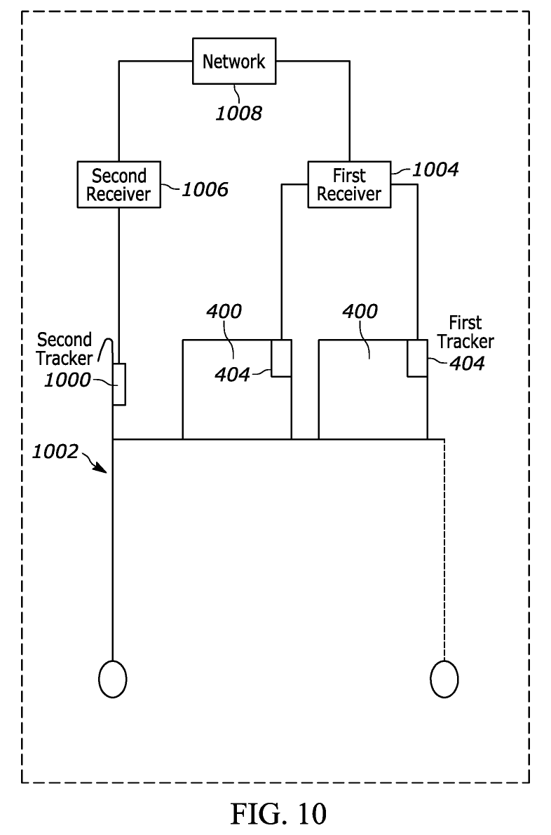

Using an example to describe the above in greater detail, FIGS. 9 and 10 illustrate asset tracking of tool trays and the carts on which they are supported to move the trays through a hospital to a target OR, either using a robot or a human. Note that robots also may be treated as assets and have corresponding tags attached to them to detect and control their motion to undertake tasks.

Commencing at state 900 in FIG. 9, first trackers or tags 404 in FIG. 10 are engaged with respective trays 400. The first trackers 404, as indicated above in reference to FIG. 7, are designed to withstand autoclave sterilization and to facilitate real-time monitoring and verification of the sterilization process using, e.g., temperature sensors and humidity sensors without compromising the protective sterile wrap.

Moving to state 910 in FIG. 9, a respective second tracker 1000 in FIG. 10 is engaged with a respective mobile cart 1002 that is used to transport the trays 400 throughout the hospital. The second trackers 1000 typically have a longer range than the first trackers 404 and may be implemented by, e.g., 4G GNSS, Wi-Fi, or other appropriate technology. In general, second tags or trackers may be attached to any hospital asset to aid in detecting the location of the asset, including robots, carts, prostheses, patient gurneys, etc. In some embodiments a second tag may be established by a label or sticker with a machine-readable code on it such as a bar code or quick response (QR) code to be read by cameras located in the hospital.

State 920 in FIG. 9 indicates that trays 400 are placed onto the cart 1002 as shown in FIG. 10. The presence of the trays and cart are detected at state 930 at locations in the hospital, which may be thought of as checkpoints, that are passed by the cart as it moves. A checkpoint may include both first reader or receivers 1004 as shown in FIG. 10 that are configured to receive information from the first tags or trackers 404 and second readers or receivers 1006 that are configured to receive information including cart ID information from the second tag or tracker 1000 of the cart 1002. Or, the first and second receivers on a cart may communicate with each other and subsequently the second receiver used to communicate all data from the cart to the network including the DB system 106. When the cart passes in proximity to a checkpoint, information from the first receiver 1004 as received from a first tag 404 is sent to a network 1008 at state 940 to which the DB system is connected to verify condition and presence of trays 400. Likewise, information from a second receiver 1006 indicating identity of the cart 1002 as received from the second tag or tracker 1000 is sent to the DB system via the network 1008. The DB system associates the detected trays 400 with the cart 1002. In one embodiment, a cart with its trays may be distinguished from another cart with its trays using the signal strength of the first trackers 404 and/or second trackers 1000 at the respective checkpoint receivers.

It may now be appreciated that the cart 1002 may be autonomous on its own or pushed by a robot, and that as the cart navigates through the hospital using, e.g., cameras, proximity sensors, and an electronic map, it passes various readers mounted on strategic locations in halls and doorways which read the tag of the cart to track in real time the location of the cart and its contents. The real time location of each asset in the hospital is logged into the database and can be accessed by hospital personnel and other robots. In this way, an OR team is kept aware of location and timing for bringing the cart.

At state 950 in FIG. 9 the location, condition, status, and other pertinent information regarding the trays and carts may be selectively displayed on a display or sent to a robot to enable a human or robot user to select trays with surgical tools of interest and obtain information relating to its location and sterilization status.

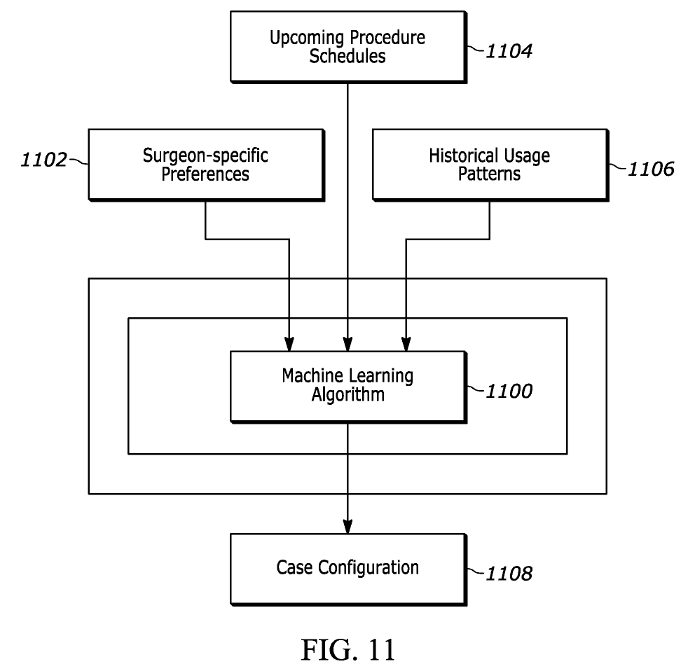

Indeed and continuing the asset-to-OR delivery process, FIG. 11 illustrates that one or more ML models 1100 may receive as input surgeon-specific preferences 1102 including particular tools or implants favored by certain surgeons or known variations in technique, upcoming procedures schedule 1104, and historical surgeon-specific and/or surgeon-independent usage patterns 1106 such as instruments and quantities used in past surgeries of various types. The ML model 1100, on the basis of its input, may output an electronic and/or human perceptible case configuration 1108.

The case configuration 1108 may include a composition of instrument trays for an upcoming surgery to ensure all needed tools are included without excess, along with recommended replenishment quantities for consumable items such as sutures, sponges, or implants. The case configuration 1108 also may include restocking instructions and alerts for any anticipated shortages. Training of the ML model 1100 may be refined using data from each surgery. The case configuration output 1108 can be used to control one or more robots to convey plural assets in the hospital to at least one operating room for use in an operation .

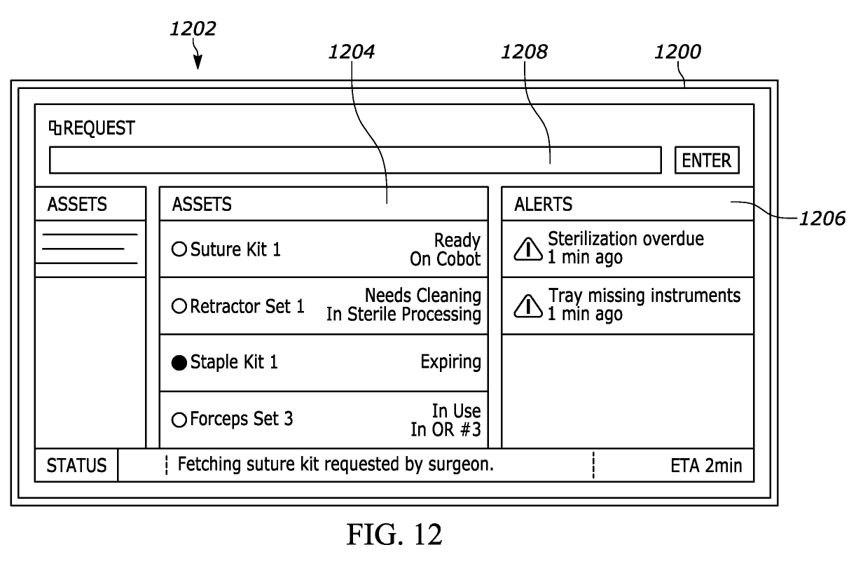

FIG. 12 illustrates a display 1200 presenting a user interface (UI) 1202 that may be presented in an OR either on a standalone display, for instance on a portable computer that a clinician may carry, or a display on a robot through which clinicians can engage with the system and monitor inventory status. The UI 1202 may include a list 1204 of assets by name, location, usage status, and replenishment needs or alerts 1206. In non-limiting examples the assets may be listed along with color-coded status icons, and an input field 1208 may be provided for clinicians to enter requests for a specific instrument or supply kit. In response a robot may fetch the requested item. Voice commands also may be accepted.

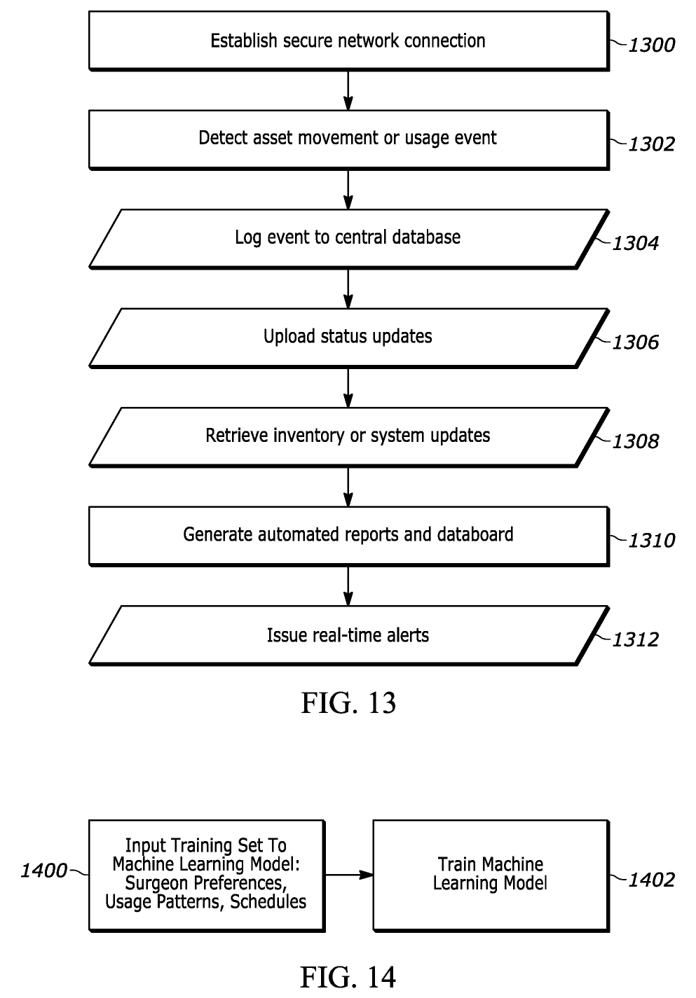

FIG. 13 illustrates data synchronization of events in the OR with the DB system. Commencing at state 1300, a robot may establish secure network communication with the DB system. Moving to state 1302, asset movement such as movement of an instrument tray, use of an instrument during a procedure, or return of an instrument for reprocessing is detected (by a camera on the robot or in the OR) and reported at state 1304 to the DB system via the hospital network. The report may include, e.g., asset ID, time, origin, destination, and usage status.

Proceeding to state 1306, asset status updates are transmitted to the DB system while at state 1308 current inventory is reported to the DB system and updates from the DB system, such as notifications that newly sterilized kits are available for pickup, are received. State 1310 indicates that usage and performance data are compiled by the DB system into automated reports, such as use frequency, tray turnaround times, and inventory consumption metrics, for use by perioperative operations management or supply chain personnel. These reports may be delivered periodically via electronic communication or displayed on a management dashboard.

If desired, at state 1312 alerts such as identification of a missing, expired, or non-sterile instrument may be presented to relevant personnel or systems.

FIG. 14 illustrates example training logic for the ML model 1100 in FIG. 11. At state 1400 input training set of surgeon preferences, historical usage patterns, and operation schedules is input the to model to train the model at state 1400. Note that like the other ML models described herein, the model 1100 may be implemented by a single model operating on heterogenous data inputs or it may be implemented by plural models each operating on its own data type and centrally managed by a management ML model.

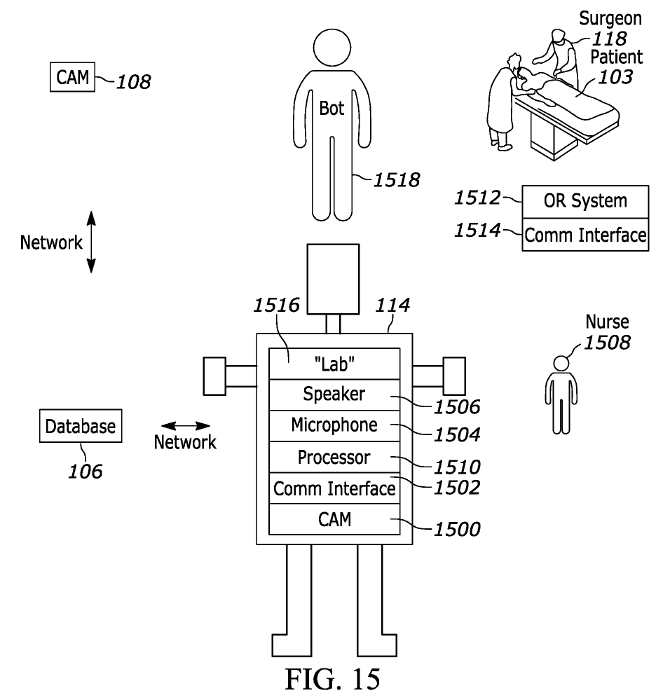

FIG. 15 illustrates example features of the robot 114 from FIG. 1 in an OR in which the surgeon 118 from FIG. 1 is operating on the patient 103. As shown, the robot 114 may include one or more onboard sensors 1500 such as a camera, LiDAR, or ultrasonic sensor to detect obstacles and sterile field boundaries for adjusting a path of the robot to avoid interfering with sterile areas. The robot 114 may include a communications interface 1502 for communicating over a network path with the DB system 106 to control the robot to move to one or more locations at which respective assets required for the operation are located according to the database system and to engage one or more of the assets for conveyance thereof; and

Also, the robot may include one or more microphones 1054 and one or more speakers 1506 for receiving voice commands from a human 1508 and for communicating audibly with the human.

With the vision system that includes the cameras 108 and 1500, data on workflow, surgeon and team actions is gathered and communicated to humans and robots. As discussed above in relation to FIG. 1, with the microphone 1504 and speaker 1506 controlled by an internal processor assembly 1510, the robot 114 can function as a command center, with which the human 1508 can talk and give commands linked to the DB system 106. The cameras and other sensors described herein record data off any machine 1512 with communication interface 1514 in the OR, including an anesthesia machine, heart rate machine, etc. The data may be imaged by the cameras and/or communicated directly from the OR machine 1512 to the DB 106. Data is also sent from the robot itself to the DB 106 such that data being collected from the robot and from the workflow as imaged by cameras and/or reported by OR machines is “meshed” together by the DB 106 to give the hospital insight on how humans use the robot and how workflow (or a robot making mistakes) can be improved.

In addition to the above, the robot 114 may function as a carrier for other task-solving systems including circuitry, reagents, and hardware establishing a laboratory 1516, such as a pharmacy lab, an infectious disease lab, tumor lab, ultrasound lab and x-ray analysis lab. For the latter example an end effector of the robot 114 may be configured as an x-ray emitter.

Completing the description of FIG. 15, the robot 114 may be a first of plural robots in the OR and may communicate with a second robot 1518 using the communication interface 1502. The ML model executed by the processor assembly 1510 for controlling the first robot 114 may communicate with the ML model controlling the second robot 1518. Among other beneficial advantages, this enables the first robot, for instance, to have downloaded from the DB system 106 patient data including scans, the type of operation to be performed and the tools and/or prostheses needed, and transmit that information to the second robot 1518 to configure the second robot to perform or otherwise assist in the operation.

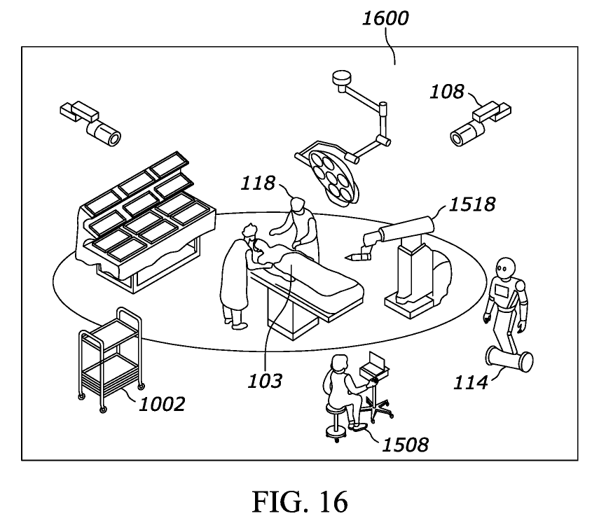

FIG. 16 depicts an example environment of the components above in an OR 1600. One or more of the cameras 108 may be strategically placed in the OR to fully capture events in the OR. Microphones borne on a robot or mounted in the OR may be used to record audio associated with the video from the cameras. If desired, data compression such as implementing ML-based event detectors (to detect the beginning and end of particular events) may be used to prune video classified as being of less importance. The data collected by the various elements described herein may be time-stamped and fused in the DB 106 for a contextual, synthesized awareness of the surgery.

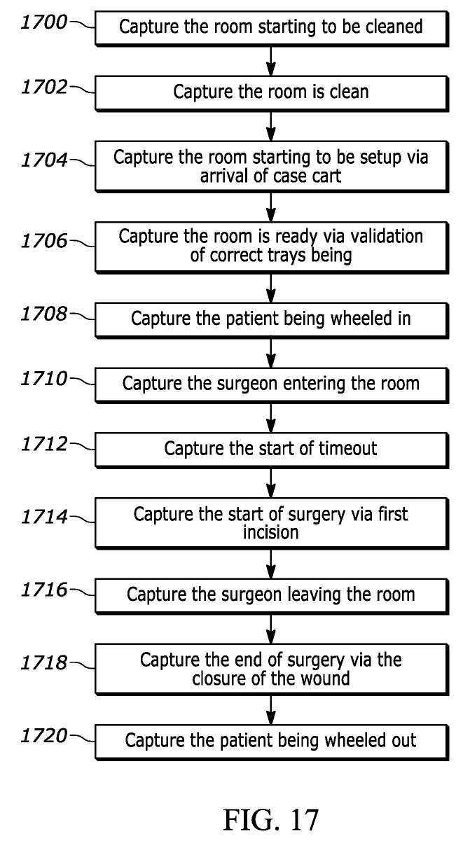

FIGS. 17-23 illustrate further principles associated with the OR theater of FIGS. 15 and 16. In particular, FIG. 17 illustrates logic for capturing images of workflow in the OR and identifying key workflow events. The logic may be executed by any one or more processor assemblies in any one or more of the systems and components discussed herein, working is cooperation with each other where appropriate via network links, with executable code and data being stored in one or more data storages such as the DB system 106.

Commencing at state 1700 in FIG. 17, prior to an operation the camera(s) 108 in FIG. 16 (including any cameras that may be included on a robot) capture still and/or video images of the OR as it is being cleaned. Moving to state 1702, the cameras continue to capture images of the OR as it is being set up for surgery as detected using machine vision recognizing arrival of the cart 1002. Note that “machine vision” may be supplemented at any state with audible analysis of sound picked up by microphones in the OR.

Moving to state 1704, the cameras continue to capture images of the OR which are analyzed using machine vision to determine, by recognizing that correct tool trays are on the cart, that the OR is ready for surgery. Note that this may include accessing the various first and second readers or receivers detecting presence of the cart with trays correlated to it as the cart with trays is wheeled into the OR, in combination with imaging the cart (including a visible code on the cart when provided).

At state 1708 the cameras capture images of the patient on a gurney being conveyed into the OR, while state 1710 indicate that the cameras capture images of the surgeon entering the OR. As before, the images are analyzed using machine vision to identify the particular workflow events being imaged, all of which are time-stamped for correlation with additional workflow-related data including audio captured by microphones, data logged by OR systems 1512 and/or the nurse 1508, and haptic feedback data as may be sensed and uploaded by end effectors of the robot 114.

State 1712 indicates identification, using machine vision implemented on the images from the OR cameras, the start of a time out during surgery, e.g., as indicated by the surgeon stepping away from the operating table. State 1714 indicates identifying the start of surgery by identifying via machine learning on the OR images the making of the first incision into the patient.

Imaging continues throughout the process to capture the complete workflow, and accordingly state 1716 indicates that the machine vision algorithm has identified, based on the imaging, the surgeon departing the OR. The patient is then closed which is detected at state 1718 and then removed from the OR which is detected at state 1720. Subsequent workflow events including OR cleanup and causing the robot to engage and return tools to the SPD also may be identified through imaging (supplemented by sound picked up by microphones if desired) to capture the entire workflow.

The workflow as identified in the logic of FIG. 17 can be analyzed by a ML model trained on prior workflows with ground truth annotation by a surgeon or other subject matter expert as to the efficacy of the particular workflow. Also, the workflow as identified in the logic of FIG. 17 can itself be used to refine training of the ML model, with a subject matter expert annotating the workflow as above as to whether the workflow was efficacious or not. Ground truth annotation as to efficacy may be a numeric standard, such as 1-5, with 1 indicating poor, 5 indicating best, and 2-4 graduating in efficacy from 1 to 5.

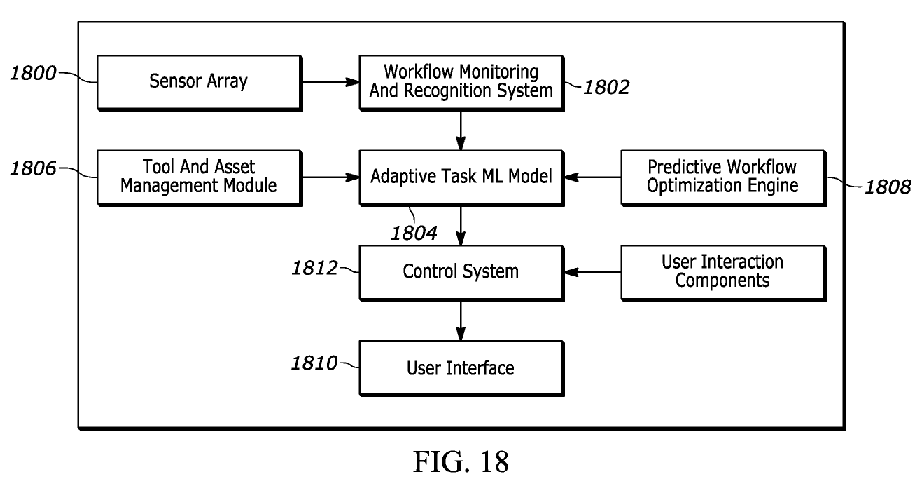

Refer now to FIG. 18, which illustrates aspects of the robot 114 consistent with disclosure above. The robot 114 may include a sensor array 1800 such as one or more cameras sending images to a workflow monitoring and recognition system 1802, which may implement the logic of FIG. 17 and which can communicate with, e.g., the DB system 106.

The recognition system 1802 communicates with an adaptive task MO model 1804 trained to indicate, based on its input, actions the robot 114 should take. The ML model 1804 may be trained on a data set that includes workflows with ground truth indications of actions the robot should take for a particular workflow, if desired for a particular surgeon. The ML model 1804 uses the identified workflow phase and may also monitor the team’s behavior and needs (for instance, detecting if a nurse’s hands are busy or if a surgeon is requesting an item) to assign or adjust tasks for the robot.

As mentioned previously in relation to FIG. 1, the robot 114 may also include a tool and asset management module 1806 that tracks surgical instruments and other assets in real-time by receiving input from sensors such as RFID readers and/or vision sensors and/or first or second presence readers or receivers to monitor instrument usage and tray inventory. Information from the tool management module 1806 is sent to both the task allocation ML model 1804 and the workflow recognition system 1802 as cues to the procedure’s progress.

The robot 114 may further include a predictive workflow ML model 1808 that is trained to output actions for the robot 114 to take including, as indicated above, surgeon-specific preferences. The model 1808 also may be trained to account for the current patient’s context and past similar surgeries, to anticipate upcoming needs or potential bottlenecks. Its predictions are sent to the task allocation ML model 1804 can also be communicated to the surgical team via, e.g., a UI 1810 as advance warnings or suggestions. The control system 1812 that actuates mechanics of the robot accordingly is activate to execute the tasks from the ML model 1804. If desired, user interaction components 1812 which may include various interfaces through which the surgical staff communicate with the robot may be used to provide real-time cues to the team and to accept commands via spoken instructions, touch inputs, or hand gestures.

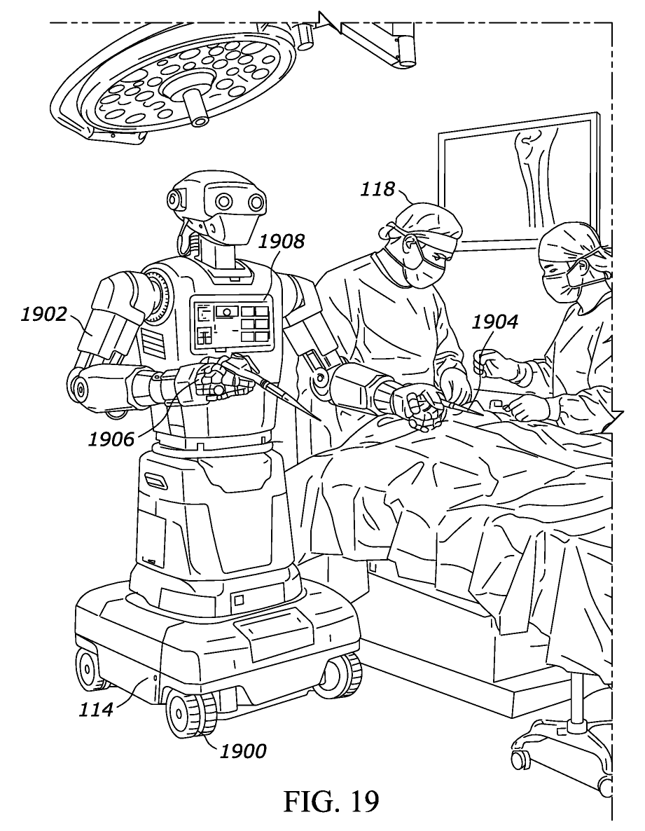

Refer now to FIG. 19. The robot 114 may take a humanoid form and may move on wheels 1900 and/or articulating legs (not shown in FIG. 19). The robot may include articulated robotic arms 1902 that are shown in FIG. 19 as handing a tool 1904 to the surgeon 118. Each arm 1902 may terminate in an end-effector 1906 that can be modular and interchangeable with other end-effectors. For instance, the end-effector can be a multi-fingered robotic hand capable of grasping surgical instruments, or a specialized tool attachment such as a gripper, clamp, suction device, drill or instrument tray holder depending on the task requirements. The robot may manipulate standard surgical tools and objects made for human use by means of fingered end-effectors with force and tactile sensors, coupled with optical cameras.

The robot also may include a touchscreen display 1908 and status indicator lights, enabling team members to receive visual information or input commands. Speakers and microphones (not shown in FIG. 19) may be included on the robot 114.

The robot 114 may be covered by a polymer skin or removable sterile drapes to allow operation in sterile environments.

Prior to a surgical procedure, the robot 114 may autonomously move to the OR using coordination with the DB 106 to ensure that all necessary personnel and tools are present and ready, helping to synchronize the team for a timely surgical start. During surgery, the robot may operate collaboratively by handing instruments, adjusting overhead lighting or other equipment, and retrieving additional supplies in concert with the human team’s workflow. The robot 114 may maintain a safe distance from the sterile field. After the procedure the robot 114 may assist with cleanup and turnover tasks, such as holding a disposal bin for used instruments, helping count instruments and sponges for audit, or preparing the room for the next surgery.

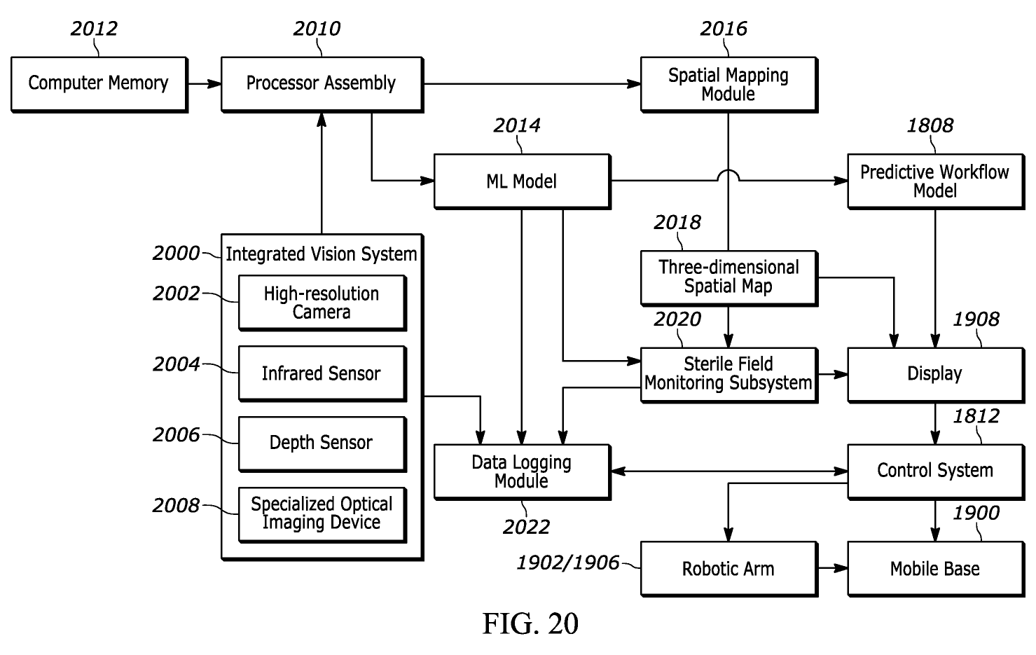

FIGS. 20-23 illustrate further principles. In addition to or in amplification of the components of an OR robot 114 described above, the robot 114 may include a vision system 2000 that can integrate images from one or more high resolution cameras 2002, one or more infrared sensors 2004, one or more depth sensors 2006, and other optical imaging device 2008. Images from the vision system 2000 may be sent to a processor assembly 2010 accessing computer memory 2012 containing data and software including ML models shown separately in FIG. 20 and/or described elsewhere herein.

With greater specificity, the processor assembly 2010 may execute a machine vision ML model 2014 to recognize objects and actions based on the output of the vision system 2000. The processor assembly 2010 may also execute a spatial mapping module 2016 to output, based on input from the vision system 2000, a three-dimensional map 2018 of the environment. The machine vision model 2014 may output signals to the predictive workflow model 1808 described previously, and may operate and share data with the mapping module 2016 such that positions of recognized objects can be overlaid onto the 3D map 2018, which may be presented on the touchscreen display 1908 along with predicted workflow from the workflow model 1808.

Additionally, a sterile field monitoring system 2020 may receive, as input, output from the machine vision model 2014 and the 3D map 2018 to control movement of the robot to avoid sterile areas unless appropriately commanded to enter them. Sterile areas may be indicated on the display 1908. Data from the sterile field monitoring system 2020, the vision system 2000, and the machine vision model 2014 may be stored in a data logging module 2022 that can be implemented by the memory 2012 and uploaded to the DB system 106.

FIG. 20 illustrates that the control system 1812 from FIG. 18 may control the arms 1902 (and end effectors 1906) of the robot and actuate the mobile base 1900 to move as appropriate based on data from the data logging module 2022, including output of the machine vision model 2014 and workflow model 1808 as may pass through the display 1908. As alluded to above in relation to FIG. 1, this enables the robot to react in real time to changes such as the presence of an obstacle or a surgical instrument drop location for retrieval of the dropped instrument by the robot and replacement thereof if desired. Replacement may be effected by the DB system 106 communicating to the robot where the nearest sterilized replacement tool or nearest tray containing a sterilized tool is so that the robot can navigate to that location and retrieve the tool or tray to the surgeon in the OR. The control system 1812 can adjust the path or pose of the robot to avoid a collision or to improve sensor viewpoint. Also, should the surgeon modify the surgery and require a different instrument or implant, the robot can scan the OR using a camera on the robot and using machine vision recognize that the required tool is missing. The robot may communicate via the hospital network with another robot to locate and bring the required instrument tray to the OR. Alternatively, the robot in the OR may depart the OR and retrieve the required instrument itself using instrument location information in the database 106.

In some embodiments, when the processor assembly identifies a condition of interest, such as an impending predicted workflow step, a detected anomaly or deviation in workflow, proximity between the robot and surgical staff, or a potential breach of a predefined sterile field boundary, the system can generate output reflecting the identified condition. Such output may include safety alerts, workflow-related notifications, or visual cues derived from the current state of the system and its representation of the surgical environment. The outputs may be communicated to users via the display 1908 which may further include an augmented reality interface configured to present spatially registered visual overlays aligned with a live view of the surgical field. For example, the AR portion of the display may highlight a sterile field boundary monitored by the sterile field monitoring subsystem 2020, emphasize the location of a tracked instrument or anatomical structure identified by the machine vision model 2014, or present a visual cue corresponding to a predicted or anomalous procedural state determined by the predictive workflow model 1808.

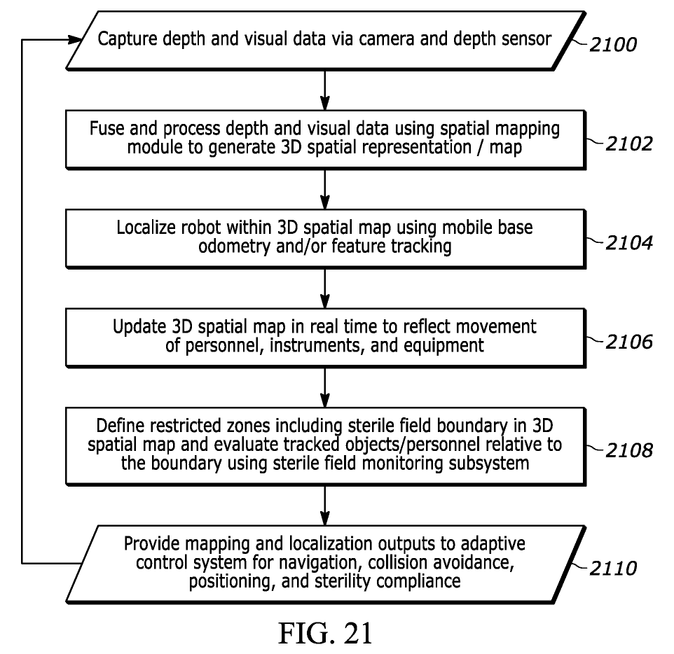

FIGS. 21-23 illustrate logic that may be executed by any one or more processor assemblies described herein, including the processor assembly 2010 in FIG. 20.

FIG. 21 illustrates example logic in example flow chart format for generating and maintaining the 3D map 2018 in FIG. 20. Commencing at state 2100, the vision system 2000 in FIG. 20 captures depth data and visual image data from the surgical environment using one or more depth sensors and cameras mounted to the robot. The captured data can be of surfaces, objects, and structural features within the operating room, including the operating table, surrounding equipment, personnel, and the robot itself.

Moving to state 2102, the captured depth and visual data are fused and processed by the spatial mapping module 2016 in FIG. 20 to generate the 3D map 2018 of the surgical environment. The spatial representation may be constructed as a point cloud, mesh, or volumetric model in which surfaces and obstacles are identified and spatially resolved.

Proceeding to state 2104, the robot determines its location in the map 2018. This can be based on feature tracking, fiducial markers, odometry from a mobile base 1900, or a combination thereof, allowing the system to continuously track its own movement relative to the mapped environment.

The logic moves from state 2104 to state 2106 to update the map 2018 in real time to reflect changes in the environment, including movement of personnel, relocation of surgical instruments, or repositioning of equipment. As objects move or are introduced into the environment, their updated positions are incorporated into the map 2018.

State 2108 indicates that one or more restricted or context-sensitive zones may be defined within the map 2018, including a predefined sterile field boundary associated with a surgical site. The system evaluates tracked objects and personnel relative to the defined boundaries to determine whether a potential breach or encroachment has occurred or is likely to occur.

Moving to state 2110, spatial mapping and localization outputs are sent to the control system 1812 shown in FIGS. 18 and 20 to move the robot as appropriate to avoid collision, safely navigate the robot, and comply with sterility requirements.

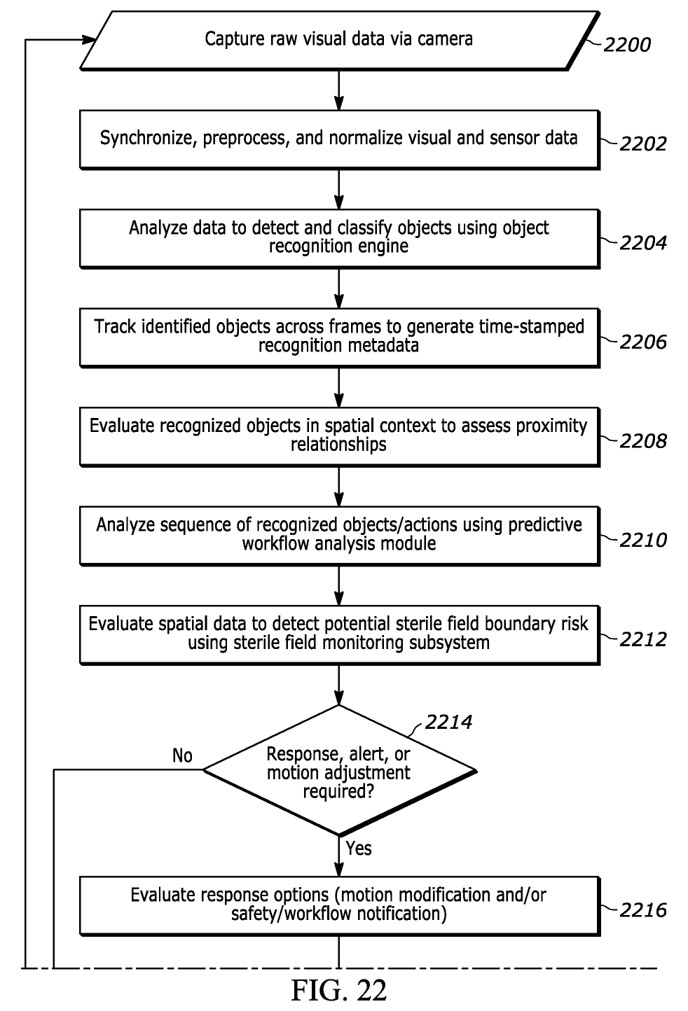

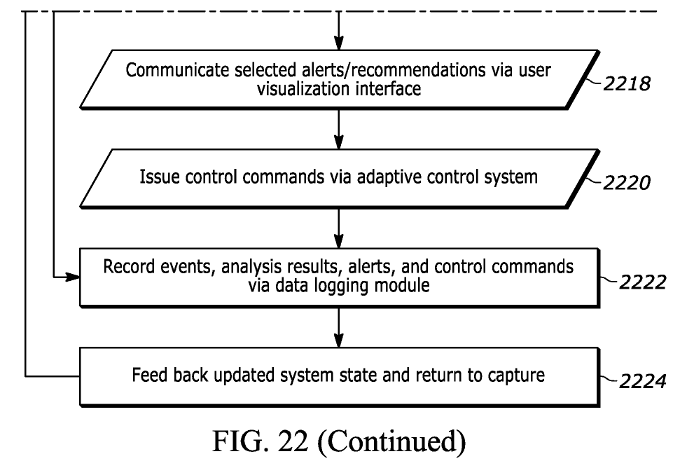

FIG. 22 illustrates machine vision-to-control of the robot. More particularly, FIG. 22 illustrates a multi-stage, closed-loop process by which visual sensing, object recognition, workflow analysis, safety monitoring, user communication, and adaptive robotic control are coordinated during perioperative operation of the robot 114. Commencing at state 2200, visual data is captured from the surgical environment optionally in combination with infrared and depth sensors. This data, at state 2202, may be synchronized, preprocessed, and normalized to account for lighting conditions, sensor alignment, and temporal consistency across multiple data streams.

Proceeding to state 2204, the data is analyzed by the machine vison model 2014 in FIG. 20 to detect and classify objects present in the surgical environment, including surgical instruments, personnel, anatomical structures, and environmental features. Moving to state 2206, objects are tracked across successive frames to determine motion trajectories, interaction events, and changes in object state, generating time-stamped recognition metadata. At state 2208 the recognized objects and tracking data are evaluated in a spatial context to assess proximity relationships between the robot 114, personnel, instruments, and anatomical structures.

The logic moves from state 2208 to state 2210 in which the workflow model 1808 from FIGS. 18 and 20 analyzes the sequence of recognized objects and actions in view of historical surgical data or predefined procedural models to determine a current procedural context, predict a forthcoming procedural step, or identify a deviation from an expected workflow. Spatial data is evaluated at state 2212 by, e.g., the sterile field monitoring system 2020 to determine whether any tracked object or person has crossed, or is likely to cross, a predefined sterile field boundary, thereby identifying a potential contamination risk.

Based on states object recognition, workflow prediction, and sterile field monitoring, decision diamond 2214 indicates that it is determined whether a response and/or alert and/or motion modification for the robot is required. If it is, the logic moves from decision diamond 2214 to state 2216 to determine which response is appropriate. State 2218 communicates alerts, recommendations, or contextual information as appropriate to surgical staff using, e.g., the display 1908.

Moving to state 2220, any necessary motion commands to move the robot are issued, including adjusting movement of the mobile base, repositioning a robotic arm or sensor, modifying speed or applied force, or altering camera orientation.

From state 2220 or from decision diamond 2214 if the decision was negative decisions, actions, and other data generated in performance of the logic of FIG. 22 are recorded at state 2222 for compliance auditing, training, and system learning purposes. At state 2224, updated system state information resulting from execution of the control commands is fed back to the integrated vision system, and the process returns to state 2200 to continue continuous monitoring, analysis, and adaptive control.

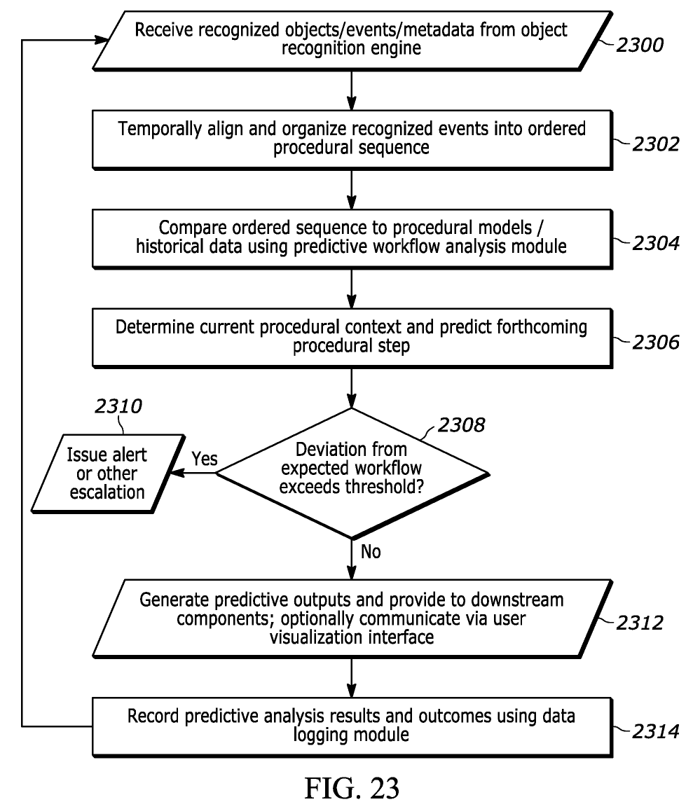

FIG. 23 illustrates details of logic executed by the predictive workflow model 1808 from FIGS. 18 and 20 for combining real-time visual recognition data with historical procedural data to determine a current procedural context, predict forthcoming procedural steps, and detect deviations from an expected surgical workflow. Commencing at state 2300, recognized objects, events, and contextual metadata generated by the machine vision model 2014 are received. The received data may include identified surgical instruments, detected personnel actions, temporal ordering of procedural events, and spatial relationships within the surgical environment.

Moving to state 2302, the received, preferably real-time recognition data is temporally aligned and organized into an ordered sequence representing recent procedural activity. This step may include filtering, weighting, or segmenting recognized events to emphasize clinically relevant actions and to reduce noise or transient detections. Then, moving to state 2304, the organized sequence of recognized events is compared against one or more stored procedural models, historical surgical datasets, or predefined workflow templates corresponding to a particular surgical procedure. While the comparison may be performed using a machine learning model, in some embodiments it may be performed using a rule-based analysis to evaluate similarity to known procedural patterns.

State 2306 indicates that based on the comparison, a current procedural context is determined to generate a prediction of at least one forthcoming procedural step, including an estimated timing or confidence level associated with the prediction if desired. Decision diamond 2308 determines whether the output of state 2306 (the observed sequence of events) deviates from an expected workflow pattern beyond a predetermined threshold, thereby identifying a potential anomaly, delay, or deviation in the procedure. When such an anomaly, delay, or deviation is identified, the deviation is classified based on one or more factors including severity, duration, confidence level, and clinical relevance at state 2310 and forwarded for downstream handling, which may include generation of a workflow-related alert, issuance of a recommendation, adjustment of system behavior, or escalation to user notification. In some embodiments, the evaluation further distinguishes between transient deviations that self-resolve and sustained deviations that warrant proactive intervention.

However, when decision diamond 2308 determines that the output of state 2306 (the observed sequence of events) does not deviate from an expected workflow pattern beyond a predetermined threshold, predictive outputs, including predicted next steps, detected deviations, or workflow-related recommendations, are generated and made available to downstream system components. In some embodiments, the predictive outputs are communicated to surgical staff via a user visualization or augmented reality interface, enabling proactive preparation or corrective action without interrupting the procedure.

State 2314 indicates that predictive analysis results, together with corresponding real-time observations and eventual procedural outcomes, are recorded to a data storage or logging module such as the computer memory 2012 in FIG. 20 and/or the DB system 106. The recorded data subsequently may be used to refine procedural models, retrain machine learning algorithms, or improve predictive accuracy in future procedures.

In some embodiments, patient faces, names, tattoos, identifiers, timestamps are removed from video and audio before data reaches secure storage. For example, a video anonymizer may be used that uses OpenCV and face detection ML to blur faces, names on charts/whiteboards, tattoos, and unique identifiers in video frames. An audio redactor can be used that employs Whisper AI and Named Entity Recognition to identify and remove patient names, birthdates, addresses, and other spoken patient-identifying information which may be replaced in the audio with silence or a beep. A metadata stripper can be used to remove detailed timestamps and other metadata that could enable re-identification. A verification engine may establish a quality control system with ML-based classification to validate anonymization completeness before permitting storage. All anonymization may occur in secure staging areas with AES-256 encryption, with identifiable data immediately deleted after successful anonymization.

Note that for each robotic task discussed above, a respective robot may be used. In other embodiments a single robot may perform two or more of the robotic tasks discussed above.

A system herein may include server and client components which may be connected over a network such that data may be exchanged between the client and server components. The client components may include one or more computing devices including mobile smart phones and other mobile devices, wearable devices such as smart glasses and smart watches, wearable devices in the form of extended reality (XR) headsets beyond smart glasses that can nonetheless present stereoscopic images thereon (such as virtual reality (VR) headsets and augmented reality (AR) headsets), display devices such as televisions (e.g., smart TVs, Internet-enabled TVs), personal computers such as laptops, desktop, and tablet computers, and still other types of client devices including but not limited to Internet of things (IoT) devices. These client devices may operate with a variety of operating environments. For example, client devices consistent with present principles may employ, as examples, Linux operating systems, operating systems from Microsoft, or a Unix operating system, or operating systems produced by Apple, Inc. or Google. These operating environments may be used to execute one or more browsing programs, such as a browser made by Microsoft, Apple, Google, or Mozilla or other browser program that can access websites and servers such as those discussed herein. The operating environments may also be used to execute other (e.g., Internet-networked) dedicated mobile applications (“apps”) that can access websites and servers over a network such as the Internet, a local intranet, or a virtual private network.

Servers and/or gateways may be used that may include one or more processors executing instructions that configure the servers to receive and transmit data over a network such as the Internet. Or a client and server can be connected over a local intranet or a virtual private network. A server or controller may be instantiated by a personal computer, mobile device, rack or blade server, etc.

Information may be exchanged over a network between client devices and servers. To this end and for security, servers and/or clients can include firewalls, load balancers, temporary storages, and proxies, and other network infrastructure for reliability and security.

As used herein, instructions may refer to computer-implemented steps for processing information in the system. Instructions can be implemented in software, firmware or hardware, or combinations thereof and include any type of programmed steps undertaken by components of the system.

A processor may be a single- or multi-chip processor that can execute logic by means of various lines such as address lines, data lines, control lines, registers, and shift registers. Moreover, any logical blocks, modules, and circuits described below can be implemented or performed with a processor/processor system such as a central processing unit (CPU), a digital signal processor (DSP), a field programmable gate array (FPGA) or other programmable logic device, an application specific integrated circuit (ASIC), discrete gate or transistor logic, discrete hardware components, or any combination thereof designed to perform the functions described herein. A processor can be implemented by a controller or state machine or a combination of computing devices.

Software modules described by way of the flow charts and user interfaces herein can include various sub-routines, procedures, etc. Without limiting the disclosure, logic stated to be executed by a particular module can be redistributed to other software modules and/or combined together in a single module and/or made available in a shareable library.

The functions and methods described below, when implemented in software, can be written in an appropriate language such as but not limited to hypertext markup language (HTML)-5, Java®/Javascript, C# or C++, and can be stored on or transmitted from a computer-readable storage medium such as a hard disk drive (HDD) or solid state drive (SSD), random access memory (RAM), read-only memory (ROM), electrically erasable programmable read-only memory (EEPROM), compact disk read-only memory (CD-ROM) or other optical disk storage such as digital versatile disc (DVD), magnetic disk storage or other magnetic storage devices including removable thumb drives, etc. A connection may establish a computer-readable medium. Such connections can include, as examples, hard-wired cables including fiber optics and coaxial wires and digital subscriber line (DSL) and twisted pair wires.

In an example, a processor system can access information over its input lines from data storage, such as a computer readable storage medium as referenced above, and/or the processor system can access information wirelessly from an Internet server by activating a wireless transceiver to send and receive data. Data typically is converted from analog signals to digital by circuitry between the antenna and the registers of the processor system when being received and from digital to analog when being transmitted. The processor system then processes the data through its shift registers to output calculated data on output lines, for presentation of the calculated data on the device, etc.

Components included in one embodiment can be used in other embodiments in any appropriate combination. For example, any of the various components described herein and/or depicted in the Figures may be combined, interchanged, or excluded from other embodiments.

"A system having at least one of A, B, and C" (likewise "a system having at least one of A, B, or C" and "a system having at least one of A, B, C") includes systems that have A alone, B alone, C alone, A and B together, A and C together, B and C together, and/or A, B, and C together.

The term “a” or “an” in reference to an entity refers to one or more of that entity. As such, the terms “a” or “an”, “one or more”, and “at least one” can be used interchangeably herein.

The term “circuit” or “circuitry” may be used in the summary, description, and/or claims. The term “circuitry” includes all levels of available integration, e.g., from discrete logic circuits to the highest level of circuit integration such as VLSI, and includes programmable logic components programmed to perform the functions of an embodiment as well as processors (e.g., special-purpose processors) programmed with instructions to perform those functions.

Network or communication interfaces can be provided on any component described herein for communication over at least one network such as the Internet, a WAN, a LAN, etc. under control of one or more processors of a processor system. These interfaces may be, without limitation, a Wi-Fi transceiver and/or wireless telephony transceiver for communicating over a wireless cellular network (e.g., operated by Verizon, T-Mobile, or AT&T), both of which are examples of a wireless computer network interface. These interfaces may also include, as additional examples, a mesh network transceiver, a wired or wireless modem or router, or other appropriate interface.

A processor system or assembly may include one or more processors acting independently or in concert with each other to execute an algorithm (e.g., the algorithms referenced herein), whether those processors are in one device or more than one device. Thus, in some specific examples, a processor system or assembly may include a single processor, while in other examples a processor system or assembly may include more than one processor. In either case, the processor system or assembly may control the devices disclosed herein to undertake present principles.

Computer memories/computer-readable storage media include disk-based or solid-state storage that are not transitory signals. Cameras may include thermal imaging camera, digital cameras such as red-green-blue cameras like webcams, infrared (IR) cameras/sensors, and/or other types of cameras or other optical sensors in communication with a processor system. Cameras may be used to gather still images (e.g., single image frames) and/or video with plural image frames consistent with present principles. Any component herein may include a Bluetooth^{®} transceiver and other Near Field Communication (NFC) element for communication with other devices using respective Bluetooth and/or NFC wireless technologies/communication standards. An example NFC element can be a radio frequency identification (RFID) element.

Touch-enabled display(s) consistent with present principles may include, for example, a capacitive or resistive touch sensing layer with a grid of electrodes for touch sensing consistent with present principles (e.g., to provide input to the graphical user interfaces (GUIs) discussed herein).

A battery may be provided for powering any devices disclosed here, as may alternating current (AC) power sources such as wall outlets. A kinetic energy harvester may also be embodied on certain devices to turn kinetic energy into power to charge the battery and/or otherwise power the device.

One or more graphics processing unit (GPUs) may also be included on a devices disclosed herein, including for AI-related processing.

A server may include at least one server processor system, at least one tangible computer readable storage medium such as disk-based or solid-state storage, and at least one network interface that, under control of the server processor system, allows for communication with the other illustrated and/or disclosed devices over the network, and indeed may facilitate communication between servers and client devices in accordance with present principles. Note that the network interface may be, e.g., a wired or wireless modem or router, Wi-Fi or Ethernet transceiver, or other appropriate interface such as, e.g., a wireless telephony transceiver.

Accordingly, in some embodiments a server may be an Internet server or an entire server “farm” of multiple servers. If desired, the server may include and perform “cloud” functions such that the devices of the system may access a “cloud” environment via the server in example embodiments.

The components shown in the figures may include some or all components described above. Any user interfaces (UI) described herein may be consolidated and/or expanded, and UI elements may be mixed and matched between UIs. UIs may be presented at a client device under control of the client device itself and/or under control of a server as remotely controlling the client device to present the UIs thereon. Also note that selectors and options on any UIs discussed herein may be selected via cursor input, touch input to a touch-enabled display on which the UI is presented, using voice input, and/or using other input methods. A graphical user interface (GUI) is one example of a UI, with it being understood that UIs may also include audible UIs and gesture-based UIs.

Present principles may employ various machine learning models, including deep learning models. Machine learning models consistent with present principles may use various algorithms trained in ways that include supervised learning, unsupervised learning, semi-supervised learning, reinforcement learning, feature learning, self-learning, and other forms of learning. Examples of such algorithms, which can be implemented by computer circuitry, include one or more neural networks, such as a convolutional neural network (CNN), a linear classifier, a recurrent neural network (RNN), and a type of RNN known as a long short-term memory (LSTM) network. Generative pre-trained transformers (GPTT) and other large language models (LLM) and more generally generative models (GM) also may be used. Support vector machines (SVM) and Bayesian networks also may be considered to be examples of machine learning models. In addition to the types of networks set forth above, models herein may be implemented by classifiers.

As understood herein, performing machine learning may therefore involve accessing and then training a model on training data to enable the model to process further data to make inferences. The training data may include one or more training sets, one or more validation sets, and one or more test sets.

An artificial neural network/artificial intelligence model trained through machine learning may thus include an input layer, an output layer, and multiple hidden layers in between that are configured and weighted to make inferences about an appropriate output.

In one particular aspect, devices and methods consistent with present principles may operate substantially as shown and described herein but may be claimed as including some but not all aspects in any intermediate generalization.

Before concluding, it is to be understood that although a software application for undertaking present principles may be vended with a device, present principles apply in instances where such an application is downloaded from a server to a device over a network such as the Internet. Furthermore, present principles apply in instances where such an application is included on a computer readable storage medium that is vended and/or provided by itself, where the computer readable storage medium is not a transitory signal and/or a signal per se.

The disclosed concepts are rooted in computer technology for computers to carry out their functions.

While particular example embodiments are herein shown and described in detail, these are not intended to be limiting and it is to be understood that the subject matter which is encompassed by the present invention is limited only by the claims.

What is claimed is:

1. An apparatus comprising:
a database system containing data collected from images and operations in at least one hospital; and
at least one computer storage storing at least one machine learning (ML) model executable by at least one processor assembly and iteratively trained on data in the database system to control one or more robots to convey plural assets in the hospital to at least one operating room for use in an operation.

2. The apparatus of claim 1, wherein the asset comprises a tray supporting surgical tools.

3. The apparatus of claim 1, wherein the asset comprises a prosthetic.

4. The apparatus of claim 1, wherein the asset comprises a patient gurney.

5. The apparatus of claim 1, wherein the ML model is configured to control the one or more robots based at least in part on an identity of a surgeon associated with the operation.

6. The apparatus of claim 1, wherein the ML model is configured to control the one or more robots based at least in part on a type of surgery the operation is.

7. The apparatus of claim 1, wherein the ML model is configured to control the one or more robots based at least in part on a time and date of the operation.

8. The apparatus of claim 1, comprising plural tracking readers mounted in the hospital to detect presence of assets and transmit indication thereof to the database system.

9. The apparatus of claim 8, wherein the tracking readers detect respective wireless tracking tags associated with respective assets.

10. The apparatus of claim 9, wherein the tracking readers comprise Bluetooth readers.

11. A method comprising:
autonomously navigating at least one robot through perioperative areas in a hospital;
using at least one machine learning (ML) model, analyzing historical asset usage data and operations to predict upcoming asset demand;
identifying and tracking assets in the hospital using an asset identification technology to obtain location data for each asset identified by the ML model for upcoming asset demand;
retrieving the assets according to ML model output and conveying the assets to an operating room using the at least one robot; and
transmitting asset usage events in the operating room to a database system.

12. The method of claim 11, comprising providing the asset usage events to the ML model to refine training of the ML model.

13. The method of claim 11, comprising verifying readiness of surgical instruments by checking sterilization status and expiration dates via the asset identification technology and presenting an alert if any required instrument is found to be missing, contaminated, or expired.

14. The method of claim 11, wherein the robot comprises one or more sensors selected from cameras, LiDAR, ultrasonic sensors, to detect obstacles and sterile field boundaries for adjusting a path of the robot to avoid interfering with sterile areas.

15. The method of claim 11, comprising identifying and tracking assets by reading a unique identifier associated with each asset using at least one of an RFID reader to detect an RFID tag, an optical scanner to read a barcode or QR code, or a computer vision system to recognize the item.

16. The method of claim 11, wherein displaying an alert for a missing, contaminated, or expired instrument comprises displaying a notification on a user interface accessible via an operating room.

17. An apparatus comprising:
at least one robot;
at least one processor assembly configured to execute at least one machine learning (ML) model to control the robot to move through a hospital to an operating room in which an operation is to be conducted;
at least one database system storing information regarding assets in the hospital, the processor assembly being configured to communicate with the database system to control the robot to move to one or more locations at which respective assets required for the operation are located according to the database system and to engage one or more of the assets for conveyance thereof; and
at least one sensing system to detect presence of the assets as the robot moves them through the hospital to the operating room.

18. The apparatus of claim 17, wherein the asset comprises a tray supporting surgical tools.

19. The apparatus of claim 17, wherein the asset comprises a prosthetic.

20. The apparatus of claim 17, wherein the ML model is configured to control the at least one or more robots based at least in part on an identity of a surgeon associated with the operation, a type of surgery the operation is, and a time and date of the operation.
